(19) United States
(12) Patent Application Publication
(10) Pub. No.: US 2006/0094755 A1
(43) Pub. Date: May 4, 2006

(54) Novel quinoline-based metal chelators as antiviral agents

(75) Inventors: Raghavan Rajagopalan, Solon, OH (US); John Sam Babu, Bay Village, OH (US)

Correspondence Address: Raghavan Rajagopalan, Ph.D.;Bioflexis, LLC, Suite 260, 11000 Cedar Avenue, Cleveland, OH, 44106, US

(73) Assignee: Bioflexis, LLC, Cleveland, OH (US)

(21) Appl. No.: 11/260,780

(22) Filed: Oct. 27, 2005

Related U.S. Application Data
(60) Provisional application No. 60/622,905, filed on Oct. 28, 2004.

(51) Int. Cl. C07D 215/38; C07D 215/12; A61K 31/47

(52) U.S. Cl. 514/311; 514/313; 546/159; 546/176

(57) ABSTRACT

This invention relates to novel quinoline-based divalent metal ion chelating ligands of Formula I, 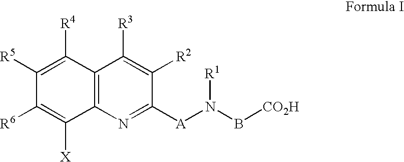
wherein A or B are independently —CR⁷R⁸, or —CH(R⁹)CH(R¹⁰). X is hydrogen, C₁-C₁₀ alkyl; —OH, or —NR¹¹R¹². R¹ to R¹² are various substituents selected to optimize the physicochemical and biological properties such as enzyme binding, tissue penetration, lipophilicity, toxicity, bioavailability, and pharmacokinetics of compounds of Formula 13. R¹ to R¹² may include, but are not limited to hydrogen, alkyl, acyl, hydroxyl, hydroxyalkyl, substituted or unsubstituted aryl, amino, aminoalkyl, alkoxyl, aryloxyl, carboxyl, halogen, alkoxycarbonyl, cyano, and other suitable electron donating or electron withdrawing groups. The compounds of the present invention are useful for inhibiting the activity of viral enzymes responsible for the proliferation of human immunodeficiency virus (HIV).

This application claims benefit of priority from Provisional Application No. 60/622,905, filed on Oct. 28, 2004.

### FIELD OF THE INVENTION

This invention relates to antiviral agents. Particularly, it relates to the compositions and methods for inhibiting the activity of HIV-integrase, a viral metalloenzyme responsible for replication cycle of human immunodeficiency virus (HIV). More particularly, the present invention discloses novel quinoline-based ligands for sequestering the divalent metal ion from the catalytic site of said enzyme.

### BACKGROUND OF THE INVENTION

It is to be noted that throughout this application various publications are referenced by Arabic numerals within brackets. Full citations for these publications are listed at the end of the specification. The disclosures of these publications are herein incorporated by reference in their entireties in order to describe fully the state of the art to which this invention pertains.

HIV infection in humans that results in AIDS is relatively a new disease as compared to other human illnesses, but is still remains the foremost health problem in the world. Although better treatment options has prolonged the survival of people infected with HIV in the US, Centers for Disease Control (CDC) estimates that nearly 800,000 people are living with AIDS in US and 40,000 new cases are reported each year. In addition to the direct impact of AIDS in HIV infected individuals, the emergence of drug resistance tuberculosis frequently seen in HIV infection has become a critical public health concern. Clearly, better treatment for HIV infection is needed to combat this chronic, debilitating deadly disease.

HIV requires three key steps in its replication inside a host cell: (a) reverse transcription of viral genomic RNA into viral cDNA by reverse transcriptase (RT); (b) integration of viral cDNA into host cell chromosomes by integrase (IN); and (c) cleavage of newly synthesized viral polypeptide by Protease into individual viral proteins during new virion assembly. The RT, Protease, and IN enzymes involved in the three key steps are made by HIV and were considered as targets for drug intervention[1]. The first generation of RT inhibitors such as AZT and its family of inhibitors as well as the recently developed protease inhibitors target the viral replication cycle before and after the viral integration step. Combination therapy using the RT and Protease inhibitors has enhanced the treatment potential of AIDS. However, these treatments do not suppress viral replication in all patients, and the virus remains active in the host cell. It is essential for integration of viral cDNA into host chromosome to form provirus in the host cells, and this process is effected by IN. Thus, molecules that can inhibit IN function are emerging as attractive candidates for new drug development against HIV [2]. The emergence of HIV strains resistant to the current anti-HIV drugs necessitates the development of new ones to combat AIDS.

IN is a metalloenzyme that exists as a dimer or tetramer having two or four catalytic sites, respectively. IN inhibitors generally can be classified as one those that target both 3′ processing and strand transfer reactions (bifunctional inhibitors) and the other that inhibit strand transfer reaction alone (ST-inhibitors). The mechanism of IN has been studied extensively, and it was found that Mg²⁺ or Mn²⁺ ion plays a key role in both the 3′-processing and in the strand transfer process [4]. Although in vitro Mg²⁺ and Mn²⁺ can equally substitute each other in enzyme function, it is well understood that Mg²⁺ plays the key role in vivo. The catalytic core domain of all IN contains the invariant amino acid triad D-D-E motif [3], and in the case of HIV-1 IN, the triad contains amino acid residues D64, D116, and E152. By analogy with DNA polymerase mediated catalysis models, it was suggested that Mg²⁺ or Mn²⁺ ion bound to this amino acid triad plays a key role in IN catalysis. Functional mutagenesis studies show that when any one of the triad residue is modified, the catalytic activity of IN is either abrogates or severely compromised [4-7]. Specifically, the divalent metal ion facilitates the hydrolysis of phosphodiester bond by increasing the electrophilicity of phosphorous upon coordination. In the same manner, by increasing the electrophilicity of phosphorous, it also increases the addition of 3′-hydroxyl of a nucleotide to make the phosphodiester bond.

There has been considerable effort in developing IN inhibitors endowed with divalent metal ion binding motifs. As shown in FIG. 1, the classes of molecules varies from simple catecholarsonium salt 1 [8] and the hydrazide 2 [9, 10] to complex steroid 5 [11] wherein the principal divalent metal ion motifs include catechols, 1,2-diols, β-diketones, o-hydroxyacids, hydrazides, quinolinols, and the like. These inhibitors also contain other pharmacophores required for anchoring the molecules in the hydrophobic pocket of the IN, and orienting the metal-binding motif properly in the catalytic site.

Much attention has been directed to the development of β-diketo compounds 6 to 11 (FIG. 2). Some of these compounds, viz. L-708, 906 (6) and L-731, 988 (8), inhibit strand transfer reaction but do not inhibit 3′ processing, while other such as 5CITEP (10) inhibit both reactions. Further structure-activity relationship (SAR) studies lead to the discovery of compounds bearing two β-diketo motifs (compound 11) that were effective in retaining both 3′-processing and strand transfer inhibition function. Although the mechanism by which these inhibitors inactivate IN function is not yet firmly established, it is commonly accepted that the β-diketo motif sequesters the divalent metal ion from the active site and inhibit enzyme catalysis.

The β-diketo compounds 6 to 11 have a major problem with respect to drug development in that the aldehydes and ketones are generally disfavored due to their propensity to react with the ε-amino group of the lysine residues in serum albumin and in other proteins [12]. This reactivity is, at best, reduces bioavailability, and at worst, may cause undesirable side effects. For example, the second generation of 5CITEP derivative compound 10 has an IC₅₀ of 20 nM in in vitro enzyme inhibition assay but its EC₅₀ is reduced to 700 nM in ex-vivo viral inhibition assay. Similar trend is observed for other β-diketo based inhibitors as well [13].

Although compounds 1-11 are endowed with Mg²⁺ or Mn²⁺ ion binding motif, these inhibitors will be able to sequester these ions from the active site only if the motifs are accessible to the enzyme-bound metal ion. For example, in the X-ray crystallographic study involving the inhibitor, 5CITEP-bound IN [14], it was revealed that the ligand does not displace the magnesium ion bounded to both Asp-64 and Asp- 116 residues in the enzyme. The lack of displacement could be attributed either to the insufficient chelating power of the β-diketo motif or to the unfavorable orientation of the inhibitor inside the active site. Nevertheless, current evidence suggests that inhibitors that bind to the active site, as well as chelate the metal ions will be better candidates than simple space-occupying competitive inhibitors wherein the metal ion binding are not in close proximity to the metal. Perhaps the most convincing evidence that Mg²⁺ or Mn²⁺ chelators based IN inhibitors are effective antiviral agents is provided by the hydroxyquinoxaline derivative 12. This compound, which lacks the β-diketo motif, has similar IC₅₀ value (0.01 μM) to 9, but substantially better in ex vivo viral inhibition with EC₅₀ of 0.004 μM [15]. This can be attributed to the presence of multiple coordination sites as indicated by structures 13a-c. Similar trend is also observed in compound 11 where there are two metal ion binding sites compared to all other β-diketo derivatives 6-10 that contain only one metal binding site. Therefore, the antiviral activity of the IN inhibitors can be substantially improved, if the probability of sequestering magnesium ion from the active site is increased by incorporating multiple Mg²⁺ or Mn²⁺ ion coordination sites in the design of novel inhibitors. Thus, there is a need to develop IN inhibitors endowed with strong divalent metal ion binding motifs that are in close proximity to the enzyme-bound metal. Ligands forming metal complexes with high stability, containing multiple coordination sites, having proper anchoring groups, and having hydrophobic residues for cell permeability are expected to be strong IN inhibitors with potent antiviral activity. Such rationally designed new generation of IN inhibitors will be useful not only in rapid therapeutic developments, but also in overcoming the current β-diketo based inhibitor resistant mutants.

### SUMMARY OF THE INVENTION

Accordingly, the present invention discloses novel chelators containing multiple divalent metal ion binding sites with similar molecular topology as the previous IN inhibitors 6-11. Specifically, the present invention relates to the novel quinoline ligands of Formula I, 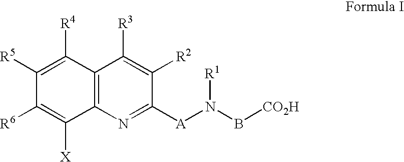
wherein A or B are independently —CR⁷R⁸, or —CH(R⁹)CH(R¹⁰). X is hydrogen, C₁-C₁₀ alkyl; —OH, or —NR¹¹R¹². R¹ to R¹² are various substituents selected to optimize the physicochemical and biological properties such as enzyme binding, tissue penetration, lipophilicity, toxicity, bioavailability, and pharmacokinetics of compounds of Formula I. R¹ to R¹² may include, but are not limited to hydrogen, alkyl, acyl, hydroxyl, hydroxyalkyl, substituted or unsubstituted aryl, amino, aminoalkyl, alkoxyl, aryloxyl, carboxyl, halogen, alkoxycarbonyl, cyano, and other suitable electron donating or electron withdrawing groups.

### BRIEF DESCRIPTION OF THE DRAWINGS

FIG. 1: HIV-1 integrase inhibitors.

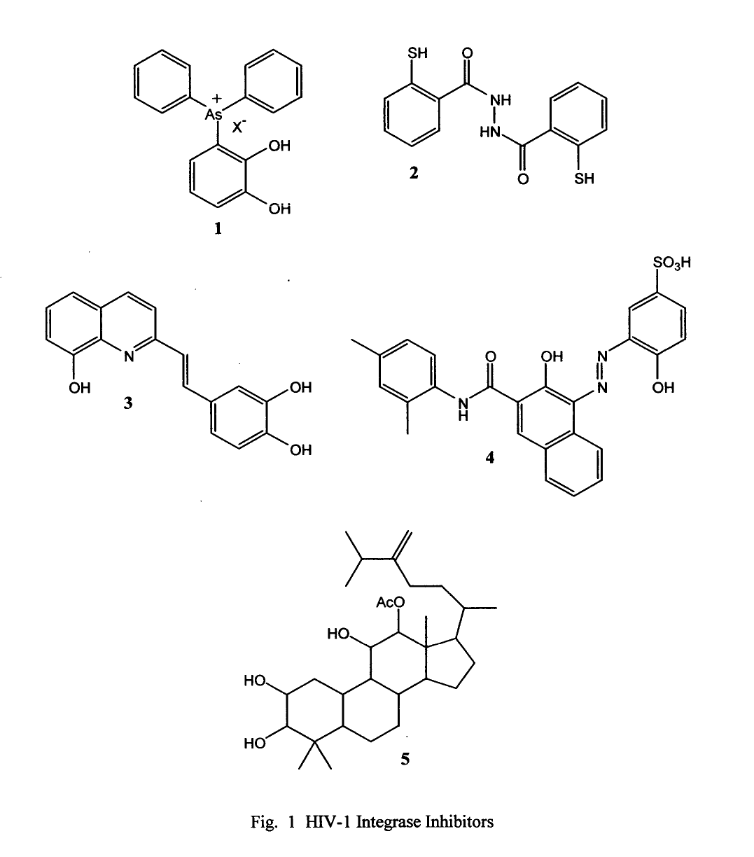

FIG. 2: β-Diketo HIV-1 integrase inhibitors.

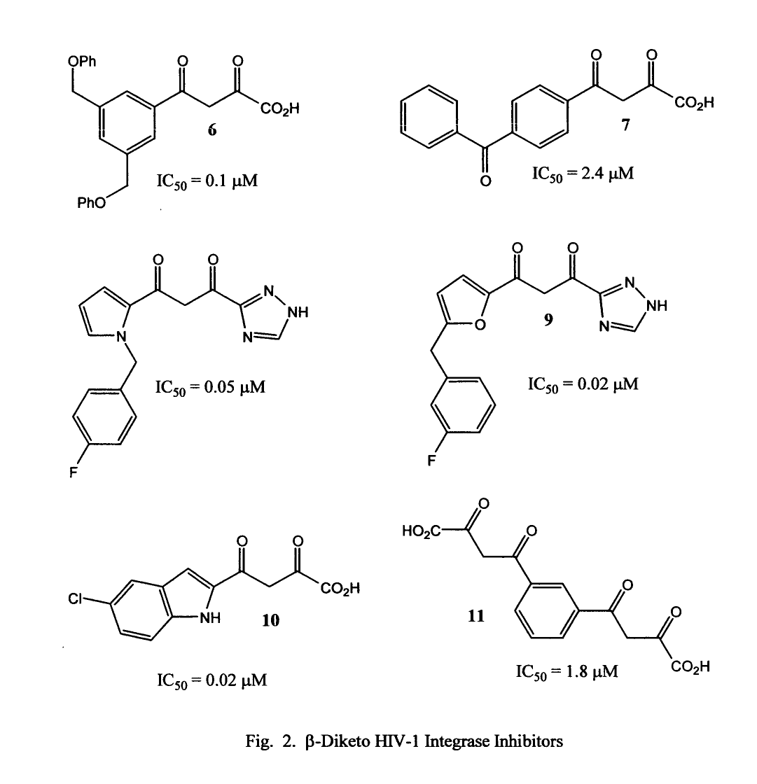

FIG. 3. Synthesis of quinoline-based ligands.

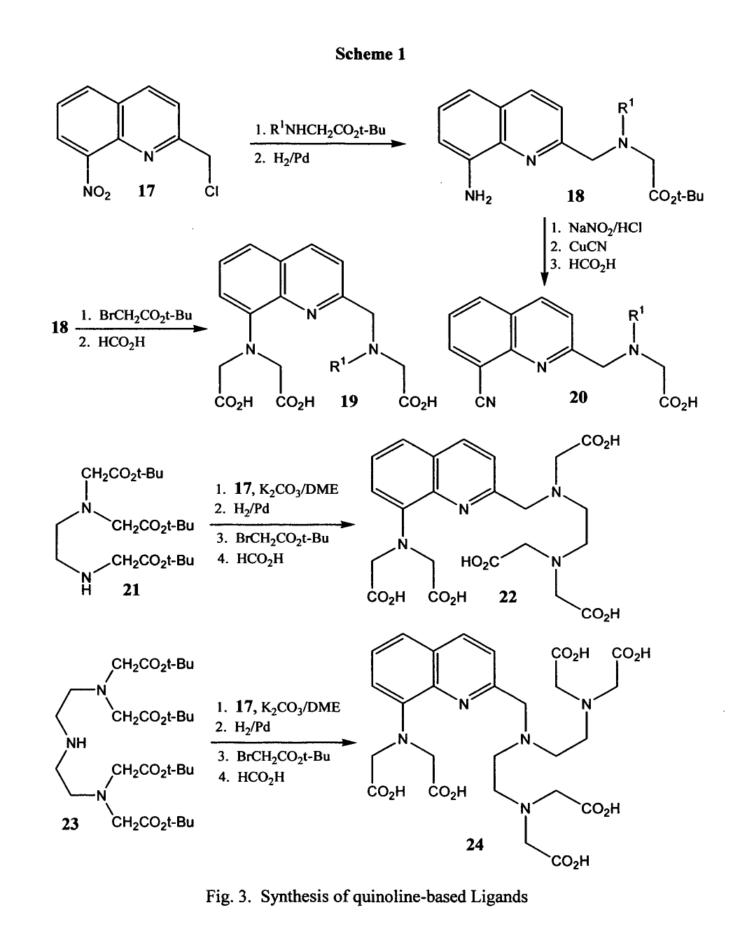

FIG. 4. Antiviral property of BFX-1001, BFX-1002, and 1003.

### DETAILED DESCRIPTION OF THE INVENTION

The present invention relates quinoline-based anti-viral compositions of Formula I, 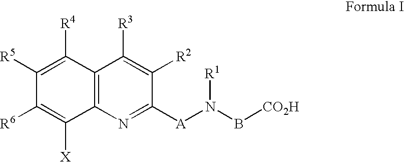
wherein A and B are independently —CR⁷R⁸, or —CH(R⁹)CH(R¹⁰). X is hydrogen, C₁ -C₁₀ alkyl; —OH, or —NR¹¹R¹². R¹ to R¹² are independently selected from the group consisting of hydrogen; C₁-C₁₀ alkyl; C₁-C₁₀ carboxyalkyl; C₁-C₁₀ alkoxyl; C₁-C₁₀ alkoxycarbonylalkyl; C₁-C₁₀ hydroxyalkyl; C₁-C₁₀ aminoalkyl; C₅-C₂₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

A preferred embodiment of the present invention is represented by Formula I, wherein A and B are —CR⁷R⁸. X is —OH. R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl. R² to R¹⁰ are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-₁₀ alkylamino, C₁-C₁₀ dialkylamino, and C₁-C₁₀ alkxoylcarbonyl.

The second preferred embodiment of the present invention is represented by Formula I, wherein A is —CH(R⁹)CH(R¹⁰). B is. —CR⁷R⁸. X is —OH. R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl. R² to R¹⁰ are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

The third preferred embodiment of the present invention is represented by Formula I, wherein A and B are —CH(R⁹)CH(R¹⁰). X is —OH. R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl. R² to R¹⁰ are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

The fourth preferred embodiment of the present invention is represented by Formula I, wherein A and B are —CR⁷R⁸. X is —OH. R¹ is selected from the group consisting of hydrogen; C₁-C₁₀ alkyl; C₁-C₁₀ carboxyalkyl. R² to R⁸ are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

The fifth preferred embodiment of the present invention is represented by Formula I, wherein A is —CH(R⁹)CH(R¹⁰). B is. —CR⁷R⁸. R¹ is selected from the group consisting of hydrogen; C₁-C₁₀ alkyl; C₁-C₁₀ carboxyalkyl. X is —OH. R² to R⁶ are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

The sixth preferred embodiment of the present invention is represented by Formula I, wherein A and B are —CH(R⁹)CH(R¹⁰). X is —OH. R¹ is selected from the group consisting of hydrogen; C₁-C₁₀ alkyl; C₁-C₁₀ carboxyalkyl. R² to R⁶ are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

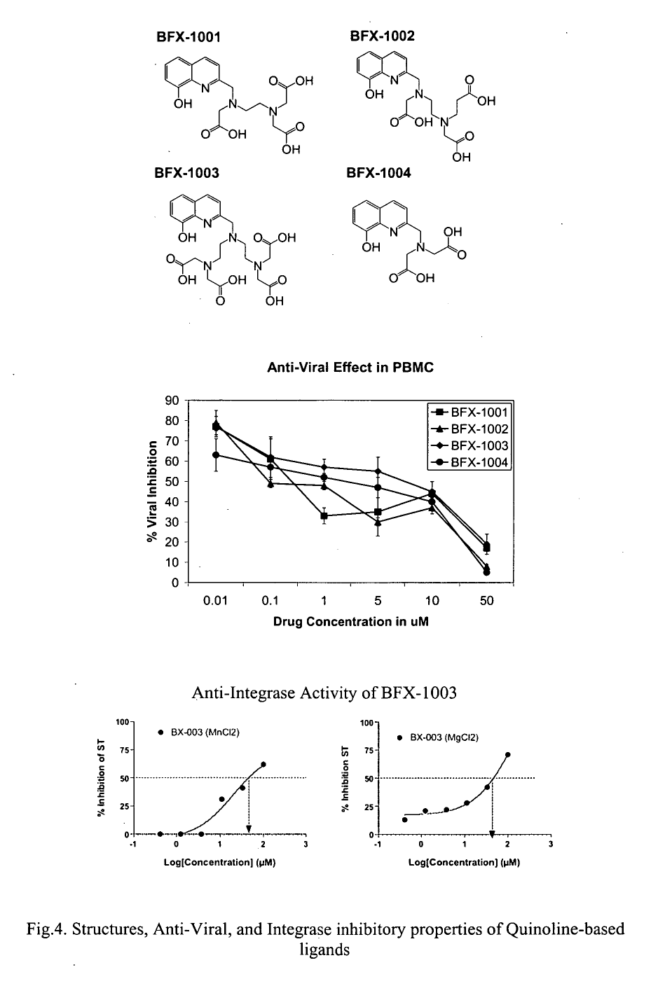

The quinoline derivatives of the present invention can be prepared by the methods well known in the art [16]. In particular, the oxine ligands 14-16 were prepared previously by Rajagopalan et al. [17], which is incorporated herein by reference in its entirety. Other functionalities such as nitro, cyano, halo, amino, or carboxymethylamino at the 8-position in 14 can be prepared by diazotization of 18 followed by displacement of diazonium group with nucleophiles such as a cyanide ion as outlined in Scheme 1 (FIG. 4). Alkylation of glycine t-butyl ester or N-alkylglycine t-butyl ester with 17 followed by functional group 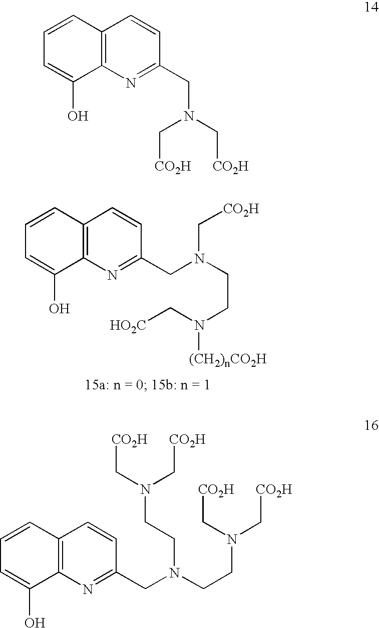
transformation of the nitro group will give analogs of 14. Similarly, alkylation of carboxyl-protected ethylenediaminetriacetic acid (21) or diethylenetriaminetetraacetic acid (23) with 17 followed by further elaboration of the nitro group will give analogs of 15 and 16. The synthesis of compounds 21 and 23 have been described by Achileful et al [18].

Compounds of the present invention may exist as a single stereoisomer or as mixture of enantiomers and diastereomers whenever chiral centers are present. Individual stereoisomers can be isolated by the methods well known in the art: diastereomers can be separated by standard purification methods such as fractional crystallization or chromatography, and enantiomers can be separated either by resolution or by chromatography using chiral columns.

Biological screening of the novel HIV inhibitors of the present invention can also be accomplished by the methods well known in the art. The 3′-processing and strand transfer events are two enzymatic functions mediated by IN and as discussed earlier, inhibitors of different classes inhibit either one or both of these events. The 3′-processing and strand transfer assays are measured in an in vitro assay using purified IN, a 21-mer duplex oligonucleotide corresponding to the U5 end of the HIV LTR sequence. The principle of the assay is described by Neamati et al. [3] and Marchand et al [13]. Briefly, 5 nM of gel purified ³²P end labeled 21-mer dsDNA oligonucleotide will be preincubated with 400 nM of HIV-1 recombinant IN (HIV-1_{NL4-3} Integrase, NIH AIDS Reagent program Catalog No:2959) for 15 min on ice in a reaction buffer (25 mM MOPS; pH7.2, 0.1 mg/mL of BSA and 14.3 mM of 2-ME). The inhibitors of the present invention are added to the reaction at various concentrations (0-100 μM) in a final volume of 10 μl and the reactions are carried out at 37° C. for 1 hr. The reactions are stopped by addition of denaturing loading dye and the samples are separated on a 20% denaturing polyacrylamide gel following standard procedures. The gels are exposed overnight, analyzed in a PhosphorImager (Molecular Dynamics, Sunnyvale, Calif.) and the densitometric analysis of the separated products in gels are determined. The 21-mer oligonucleotide is reduced in size to 19-mer following 3′-processing. The strand transfer products are larger than 21-mer and are distinguished from 3′-processing products in the same gel. The 3′ processing and strand transfer products in each lane are quantified and are expressed as a fraction of the total radioactivity. The percentage of inhibition is calculated using control lane having no inhibitors. The IN enzyme function is catalyzed by either Mg²⁺ or Mn²⁺ and the metal chelating ability of the inhibitors of the present invention will be determined in the presence of various concentrations of Mg²⁺ or Mn²⁺ (0-15 mM) in the reaction buffer.

The novel compounds of the present invention can be further evaluated for their ability to inhibit viral replication in ex-vivo assays. Most common of these include determining the viral replication in either purified human CD4+ T cell blasts infected with HIV in the presence or absence of various concentrations of inhibitors or HIV infected MT4 cell line treated with different concentration of inhibitors. A standard laboratory method in screening for inhibitors against HIV in biological assays involves the use of recombinant HIV strain that can replicate only in the supporting complementing cell line. This model system allows the examination of HIV viral replication in a biologically contained manner and is suitable for inhibitor screening. The method is described briefly below. The recombinant HIV-1 strain (HIV-1 MC99IIIBΔTat-Rev; NIH Aids Reagent Program, catalog No: 1943) is genetically engineered to replicate only in supporting recombinant cell lines (CEM-TART Cells, NIH Aids Reagent Program catalog No 1944). The construction of the recombinant mutant virus, the supporting cell line and the biological assay are described in detail elsewhere [19]. The recombinant HIV-1 strain lacks the Tat and Rev gene and infectious progeny of the virus was initially generated by transfecting viral DNA into the supporting recombinant CEM-TART cells that contain the viral Tat and Rev genes. The recombinant viral progeny is capable of infecting wide variety of cells but can undergo replication only in the supporting CEM-TART cells. This model system allows the examination of HIV viral replication in a biologically contained manner. The inhibitors of the present invention can be added to the CEM-TART cells at various concentrations before or after infection with infectious progeny of HIV-1 MC99IIIBΔTat-Rev at various time periods. The extent of viral replication can be determined by measuring the soluble viral p24 protein present in the culture supernatant collected at 24, 48, 72 and 96-hr post infection using commercial ELISA kits. Compounds 15a, 15b, and 16 of the present invention exhibited IC₅₀ of 9.6 μM, 10.2 μM, and 19.3 μM respectively in a viral inhibition assay, as shown in FIG. 4.

The compounds of the present invention can be administered in the pure form, as a pharmaceutically acceptable salt derived from inorganic or organic acids and bases, or as a pharmaceutically ‘prodrug.’ The pharmaceutical composition may also contain physiologically tolerable diluents, carriers, adjuvants, and the like. The phrase “pharmaceutically acceptable” means those formulations which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well-known in the art, and are described by Berge et al. [20]. Representative salts include, but are not limited to acetate, adipate, alginate, citrate, aspartate, benzoate, benzenesulfonate, chloride, bromide, bisulfate, butyrate, camphorate, camphor sulfonate, gluconate, glycerophosphate, hemisulfate, heptanoate, hexanoate, fumarate, maleate, succinate, oxalate, citrate, hydrochloride, hydrobromide, hydroiodide, lactate, maleate, nicotinate, 2-hydroxyethansulfonate (isothionate), methane sulfonate, 2-naphthalene sulfonate, oxalate, palmitoate, pectinate, persulfate, 3-phenylpropionate, picrate, pivalate, propionate, tartrate, phosphate, glutamate, bicarbonate, p-toluenesulfonate, undecanoate, lithium, sodium, potassium, calcium, magnesium, aluminum, ammonium, tetramethyl ammonium, tetraethylammonium, trimethylammonium, triethylammonium, diethylammonium, and the like.

The pharmaceutical compositions of this invention can be administered to humans and other mammals enterally or parenterally in a solid, liquid, or vapor form. Enteral route includes, oral, rectal, topical, buccal, and vaginal administration. Parenteral route intravenous, intramuscular, intraperitoneal, intrasternal, and subcutaneous injection or infusion. The compositions can also be delivered through a catheter for local delivery at a target site, via an intracoronary stent (a tubular device composed of a fine wire mesh), or via a biodegradable polymer. The compositions can also be delivered via an implantable drug delivery devices such as micro miniature mechanical pumps, osmotic pumps, or other similar kind of reservoirs.

The active compound is mixed under sterile conditions with a pharmaceutically acceptable carrier along with any needed preservatives, exipients, buffers, or propellants. Opthalmic formulations, eye ointments, powders and solutions are also contemplated as being within the scope of this invention. Actual dosage levels of the active ingredients in the pharmaceutical formulation can be varied so as to achieve the desired therapeutic response for a particular patient. The selected dosage level will depend upon the activity of the particular compound, the route of administration, the severity of the condition being treated, the sensitivity of the target lesions, and prior medical history of the patient being treated. However, it is within the skill of the art to start doses of the compound at levels lower than required to achieve the desired therapeutic effect and to increase it gradually until optimal therapeutic effect is achieved. The total daily dose of the compounds of this invention administered to a human or lower animal may range from about 0.0001 to about 1000 mg/kg/day. For purposes of oral administration, more preferable doses can be in the range from about 0.001 to about 5 mg/kg/day. If desired, the effective daily dose can be divided into multiple doses for purposes of administration; consequently, single dose compositions may contain such amounts or submultiples thereof to make up the daily dose.

The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated, the severity of the disorder; sensitivity of the disorder; activity of the specific compound employed; the specific composition employed, age, body weight, general health, sex, diet of the patient; the time of administration, route of administration, and rate of excretion of the specific compound employed, and the duration of the treatment. The compounds of the present invention may also be administered in combination with other drugs if medically necessary.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Prolonged absorption of the injectable pharmaceutical form can be brought about by the use of agents delaying absorption, for example, aluminum monostearate and gelatin. Proper fluidity can be maintained, for example, by the use of coating materials such as lecithin, by the maintenance of the required particle size in the case of dispersions, and by the use of surfactants. In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration include powders, sprays, ointments and inhalants. Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

The present invention also provides pharmaceutical compositions that comprise compounds of the present invention formulated together with one or more non-toxic pharmaceutically acceptable carriers. Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art [21].

The compounds of the present invention can also be administered to a patient in the form of pharmaceutically acceptable ‘prodrugs.’ The term “pharmaceutically acceptable prodrugs” as used herein represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs of the present invention may be rapidly transformed in vivo to the parent compound of the above formula, for example, by hydrolysis in blood. A thorough discussion is provided in the literature [22, 23].

The Examples presented below describe preferred embodiments and utilities of the invention and are not meant to limit the invention unless otherwise stated in the claims appended hereto. The description is intended as a non-limiting illustration, since many variations will become apparent to those skilled in the art in view thereof. It is intended that all such variation within the scope and spirit of the appended claims be embraced thereby. Changes can be made in the composition, operation, and arrangement of the method of the present invention described herein without departing from the concept and scope of the invention as defined in the claims.

### EXAMPLE 1

### Preparation of Inhibitor 19

Step 1. A mixture of 2-chloromethyl-8-nitropyridine, (10 mmol), sarcosine t-butyl ester (11 mmol) and finely ground anhydrous potassium carbonate (30 mmol) in ethylene glycol dimethyl ether (DME) (20 mL) is heated under reflux for 8 hours. The reaction mixture is filtered hot and solid is washed with 30 mL of DME. The filtrate is evaporated in vacuo and the crude product is purified by recrystallization or chromatography to give 2-(N-methyl-N-t-butoxycarbonylmethyl)methyl-8-nitroquinoline.

Step 2. A solution of the nitro compound (10 mmol) from Step 1 is dissolved in methanol and carefully treated with 10% Pd—C under a gentle flow of argon. Thereafter, the heterogeneous mixture is hydrogenated at about 3.5 atm (about 50 psi) for 4 hours. The reaction mixture is filtered through Celite and the filtrate evaporated in vacuo. The crude product is purified by recrystallization or chromatography to give 8-amino-2-(N-methyl-N-t-butoxycarbonylmethyl)methylquinoline.

Step 3. A mixture of the amine (10 mmol) from Step 2, t-butyl bromoacetate (21 mmol), and finely ground anhydrous potassium carbonate (30 mmol) in ethylene glycol dimethyl ether (DME) (20 mL) is heated under reflux for 6 hours. The reaction mixture is filtered hot and solid is washed with 30 mL of DME. The filtrate is evaporated in vacuo and the crude product is purified by recrystallization or chromatography to give pure 8-[N,N-bis(t-butoxycarbonyl)methyl]amino-2-(N-methyl-N-t-butoxycarbonylmethyl)methylquinoline.

Step 4. A solution of the tri-t-butylester (10 mmol) from Step 3 in 96% formic acid is heated to boiling and then kept at ambient temperature 16 hours. The solution is evaporated in vacuo to give the desired triacid inhibitor, 8-N,N-bis(carboxymethyl)amino-2-(N-methyl-N-carboxymethyl)methylquinoline.

### EXAMPLE 2

### Preparation of Inhibitor 20

Step 1. A mixture of the amino compound (10 mmol) from Example 1, Step 2 and sodium nitrite (15 mmol) in methanol is cooled to 0° C. and carefully treated with 1M HCl (20 mL) added dropwise under inert atmosphere. Thereafter, the mixture is stirred at 0° C. for 30 minutes. Thereafter cuprous cyanide (12 mmol) in water (10 mL) is added to the reaction mixture and stirred at ambient temperature for one hour. The reaction mixture is poured onto water and extracted with methylene chloride. The organic layer is separated, washed copiously with water, dried over anhydrous sodium sulfate, filtered, and the filtrate evaporated in vacuo. The residue is purified by chromatography or recrystallization to give 8-cyano-2-(N-methyl-N-t-butoxycarbonylmethyl)methylquinoline.

Step 2. A solution of the di-t-butylester (10 mmol) from Step 1 in 96% formic acid is heated to boiling and then kept at ambient temperature 16 hours. The solution is evaporated in vacuo to give the desired diacid inhibitor, 8-cyano-2-(N-methyl-N-carboxymethyl)methyl-quinoline, which is purified by chromatography or recrystallization.

### EXAMPLE 3

### Preparation of Inhibitor 22

Step 1. A mixture of 2-chloromethyl-8-nitropyridine, (10 mmol), the tri-t-butyl ester 21 (11 mmol) and finely ground anhydrous potassium carbonate (30 mmol) in ethylene glycol dimethyl ether (DME) (20 mL) is heated under reflux for 8 hours. The reaction mixture is filtered hot and solid is washed with 30 mL of DME. The filtrate is evaporated in vacuo and the crude product is purified by recrystallization or chromatography.

Step 2. Hydrogenation of the nitro compound (10 mmol) from Step 1 is carried out in the same manner as in Example 1, Step 2. The crude product is purified by recrystallization or chromatography.

Step 3. Alkylation of the amine (10 mmol) from Step 2, with t-butyl bromoacetate is carried out in the same manner as in Example 1, Step 3. The crude product is purified by recrystallization or chromatography.

Step 4. A solution of the penta-t-butylester (10 mmol) from Step 3 in 96% formic acid is heated to boiling and then kept at ambient temperature 16 hours. The solution is evaporated in vacuo to give the desired pentaacid inhibitor 22, which is purified by crystallization or chromatography.

### EXAMPLE 4

### Preparation of Inhibitor 24

Step 1. A mixture of 2-chloromethyl-8-nitropyridine, (10 mmol), the tetra-t-butyl ester 23 (11 mmol) and finely ground anhydrous potassium carbonate (30 mmol) in ethylene glycol dimethyl ether (DME) (20 mL) is heated under reflux for 8 hours. The reaction mixture is filtered hot and solid is washed with 30 mL of DME. The filtrate is evaporated in vacuo and the crude product is purified by recrystallization or chromatography.

Step 2. Hydrogenation of the nitro compound (10 mmol) from Step 1 is carried out in the same manner as in Example 1, Step 2. The crude product is purified by recrystallization or chromatography.

Step 3. Alkylation of the amine (10 mmol) from Step 2, with t-butyl bromoacetate is carried out in the same manner as in Example 1, Step 3. The crude product is purified by recrystallization or chromatography.

Step 4. A solution of the hexa-t-butylester (10 mmol) from Step 3 in 96% formic acid is heated to boiling and then kept at ambient temperature 16 hours. The solution is evaporated in vacuo to give the desired hexaacid inhibitor 24, which is purified by crystallization or chromatography.

### REFERENCES

1. Cos, P. et al. Plant substances as anti-HIV agents selected according to their putative mechanism of action. J. Nat. Prod. 2004, 67, 284-293.
2. Craigie, R. HIV Integrase, a brief overview from chemistry to therapeutics. J. Biol. Chem. 2001, 276, 23213-23216.
3. Neamati, N., Zhaiwei, L., Karki, R. G., Orr, A., Cowansage, K., Strumberg, D., Pais, C. G. G., Voigt, J. H., Nicklaus, M. C., Winslow, H. E., Zhao, H., Turpin, J. A., Yi, J., Skalka, A. M., Burke, T. R., Pommier, Y. Metal-dependent inhibition of HIV-1 integrase. J. Med. Chem. 2002, 45, 5661-5670.
4. Kulkosky, J., Jones, K. S., Katz, R. A., Mack, J. P., and Skalka, A. M. Mol. Cell. Biol. 1992, 12, 2331-2338.
5. Engelman, A., and Craigie, R. J. Virol. 1992, 66, 6361-6369.
6. Leavitt, A. D., Shiue, L., and Varmus, H. E. J. Biol. Chem. 1993, 268, 2113-2119.
7. VanGent, D. C., Groeneger, A. A., and Plasterk, R. H. Proc. Natl. Acad. Sci. U.S.A. 1992, 89, 9598-9602.
8. Molteni, V., Greenwald, J., Rhodes, D., Hwang, Y., Kwiatkowski, W., Bushman, F. D., Siegel, J. S. Acta Crystallogr. D Biol. Cyrstallorgr. 2001, 57, 536-544.
9. Neamati, N., Hong, H., Owen, J. M., Sunder, S., Winslow, H. E., Zhao, H., Burke, Milne, G. W., Pommier, Y. J. Med. Chem. 1998, 41, 3202-3209.
10. Zhao, H., Neamati, N., Sunder, S., Hong, H., Wand, S. J. Med. Chem. 1998, 41, 3202-3209.
11. Singh, S. B., Zink, D. L., Hazuda, D. J., Felock, P. J., Polishook, J. D., Dombrowski, A. W. PCT Int. Appl. 2000, WO 00/36132.
12. Kresge, K. Integrase Inhibitor Update. American Foundation of AIDS Research 2002. Reprinted from Treatment Directory located in the website: amf.AR.org.
13. Marchand, C. Zhang, X., Pais, G. C. G., Cowansage, K., Neamati, N., Burke, T. R., and Pommier, Y. Structural determinants for HIV-1 inhibition by β-diketoacids. J Biol. Chem. 2002, 277, 12596-12603.
14. Goldgur, Y., Craigie, R., Cohen, G. H., Fujiwara, T., Yoshinaga, T., Fujishita, T., Sugimoto, H., Endo, T., Murai, H., Davies, D. R. Proc. Natl. Acad. Sci. USA 1999, 96, 13040-13043.
15. Yoshinaga, T. et al. In vitro activity of a new HIV- 1 integrase inhibitor in clinical development. Presented at the 9^{th} conference on retroviruses and opportunistic infections. Seattle, Wash. 2002.
16. Weissberger, The Chemistry of Quinolines.
17. Rajagopalan, R. et al. Quinoline ligands and metal complexes for diagnosis and therapy. U.S. Pat. No. 6,277,841; 2001.
18. Achilefu, S. et al. JOC
19. Chen, H., Boyle, T. J., Malim, M. H., Cullen, B. R., and Lyerly, H. K. Proc. Natl. Acad. Sci. USA 1992, 89, 7678-7682.
20. S. M. Berge et al. J. Pharmaceutical Sciences 1977, 66, 1 et seq.
21. T. Higuchi and V. Stella. Pro-drugs as Novel Delivery Systems, V. 14. A.C.S. Symposium Series, 1987.
22. Edward B. Roche (Ed.). Bioreversible Carriers in Drug Design. American Pharmaceutical Association and Pergamon Press: New York 1987.
23. Prescott, Ed., Methods in Cell Biology, Volume XIV, pp. 33 et seq. Academic Press, New York, 1976.

What is claimed is:

1. A compound of Formula I, 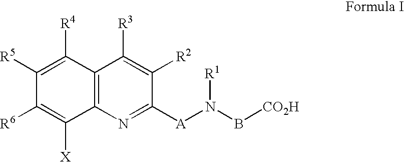
wherein A and B are independently —CR⁷R⁸ or —CH(R⁹)CH(R¹⁰); X is C₁-C₁₀ alkyl or —NR¹¹R¹²; R¹ to R¹² are independently selected from the group consisting of hydrogen; C₁-C₁₀ alkyl; C₁-C₁₀ carboxyalkyl; C₁-C₁₀ alkoxyl; C₁-C₁₀ alkoxycarbonylalkyl; C₁-C₁₀ hydroxyalkyl; C₁-C₁₀ aminoalkyl; C₅-C₂₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

2. The compound of claim 1, wherein A and B are —CR⁷R⁸; X is —NR¹¹R¹²; R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl; R² to R⁸, R¹¹, and R¹² are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

3. The compound of claim 1, wherein A is —CH(R⁹)CH(R¹⁰); B is —CR⁷R⁸; X is —NR¹¹R¹²; R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl; R² to R¹² are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

4. The compound of claim 1, wherein A and B are —CH(R⁹)CH(R¹⁰); X is —NR¹¹R¹²; R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl; R² to R⁶ and R⁹ to R¹² are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

5. The compound of claim 1, wherein A and B are —CR⁷R⁸; X is —NR¹¹R¹²; R¹ is selected from the group consisting of hydrogen; C₁-C₁₀ alkyl; C₁-C₁₀ carboxyalkyl; R² to R⁸, R¹¹, and R¹² are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

6. The compound of claim 1, wherein A is —CH(R⁹)CH(R¹⁰); B is —CR⁷R⁸; X is —NR¹¹R¹²; R¹ is selected from the group consisting of hydrogen; C₁-C₁₀ alkyl; C₁-C₁₀ carboxyalkyl; R² to R¹² are independently selected from the group consisting of C₁-C₁₀ alkyl; C₁ -C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

7. The compound of claim 1, wherein A and B are —CH(R⁹)CH(R¹⁰); X is —NR¹¹R¹²; R¹ is selected from the group consisting of hydrogen; C₁-C₁₀ alkyl; C₁-C₁₀ carboxyalkyl; R² to R⁶ and R⁹ to R¹² are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

8. The compound of claim 5, wherein A and B are —CR⁷R⁸; X is —NR¹¹R¹²; R¹, R¹¹, and R¹² are carboxymethyl; R² to R⁸ are hydrogens.

9. The compound of claim 6, wherein A is —CH(R⁹)CH(R¹⁰); B is —CR⁷R⁸; X is —NR¹¹R¹²; R¹, R¹¹, and R¹² are carboxymethyl; R² to R⁶, R⁹, and R¹⁰ are hydrogens.

10. The compound of claim 7, wherein A and B are —CH(R⁹)CH(R¹⁰); X is —NR¹¹R¹²; R¹, R¹¹, and R¹² are carboxymethyl; R² to R⁹ and R¹⁰ are hydrogens.

11. A method of treating viral infection comprising administering to an individual an effective amount of compound of Formula I, 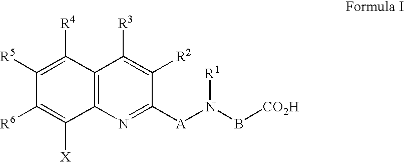
wherein A and B are independently —CR⁷R⁸ or —CH(R⁹)CH(R¹⁰); X is hydrogen, C₁-C₁₀ alkyl, —OH, or —NR¹¹R¹²; R¹ to R¹² are independently selected from the group consisting of hydrogen; C₁-C₁₀ alkyl; C₁-C₁₀ carboxyalkyl; C₁-C₁₀ alkoxyl; C₁-C₁₀ alkoxycarbonylalkyl; C₁-C₁₀ hydroxyalkyl; C₁-C₁₀ aminoalky
1; C₅-C₂₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

12. The method of claim 11, wherein A and B are —CR⁷R⁸; X is hydrogen, C₁-C₁₀ alkyl, —OH, or —NR¹¹R¹²; R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl; R² to R⁸, R¹¹, and R¹² are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

13. The method of claim 11, wherein A is —CH(R⁹)CH(R¹⁰); B is —CR⁷R⁸; X is hydrogen, C₁-C₁₀ alkyl, —OH, or —NR¹¹R¹²; R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl; R² to R¹² are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

14. The method of claim 11, wherein A and B are —CH(R⁹)CH(R¹⁰); X is hydrogen, C₁-C₁₀ alkyl, —OH, or —NR¹¹R¹²; R¹ is selected from the group consisting of hydrogen; C₁-C₁₀ alkyl; C₁-C₁₀ carboxyalkyl; C₁-C₁₀ hydroxyalkyl; and C₁-C₁₀ aminoalkyl; R² to R⁶ and R⁹ to R¹² are independently selected from the group consisting of hydrogen; C₁-C₁₀ alkyl; C₁-C₁₀ alkoxyl; C₅-C₂₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ aryloxyalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₂₀ arylalkoxyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl.

15. The method of claim 11, wherein A and B are —CR⁷R⁸; X is hydrogen, C₁-C₁₀ alkyl, —OH, or —NR¹¹R¹²; R¹ is selected from the group consisting of hydrogen; C₁-C₁₀ alkyl; C₁-C₁₀ carboxyalkyl; R² to R⁸, R¹¹, and R¹² are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

16. The method of claim 11, wherein A is —CH(R⁹)CH(R¹⁰); B is —CR⁷R⁸; X is hydrogen, C₁-C₁₀ alkyl, —OH, or —NR¹¹R¹²; R¹ is selected from the group consisting of hydrogen; C₁-C₁₀ alkyl; C₁-C₁₀ carboxyalkyl; R² to R¹² are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

17. The method of claim 11, wherein A and B are —CH(R⁹)CH(R¹⁰); X is hydrogen, C₁-C₁₀ alkyl, —OH, or —NR¹¹R¹²; R¹ is selected from the group consisting of hydrogen; C₁-C₁₀ alkyl; C₁-C₁₀ carboxyalkyl; R² to R⁶ and R⁹ to R¹² are independently selected from the group consisting of C₁-C₁₀ alkyl; C₁-C₁₀ alkoxyl; C₅-C₂₀ arylalkyl unsubstituted or substituted with C₁-C₁₀ alkyl, hydroxyl, halo, trihaloalkyl, carboxyl, and amino; C₅-C₂₀ aryloxyalkyl unsubstituted or substituted with C₁-C₁₀ alkyl, hydroxyl, halo, trihaloalkyl, carboxyl, and amino; C₅-C₂₀ arylalkoxyl unsubstituted or substituted with C₁-C₁₀ alkyl, hydroxyl, halo, trihaloalkyl, carboxyl, and amino.

18. The method of claim 15, wherein A and B are —CR⁷R⁸; X is hydrogen, C₁-C₁₀ alkyl, —OH, or —NR¹¹R¹²; R¹, R¹¹, and R¹² are carboxymethyl; R² to R⁸ are hydrogens.

19. The method of claim 16, wherein A is —CH(R⁹)CH(R¹⁰); B is —CR⁷R⁸; X is hydrogen, C₁-C₁₀ alkyl, —OH, or —NR¹¹R¹²; R¹, R¹¹, and R¹² are carboxymethyl; R² to R¹⁰ are hydrogens.

20. The method of claim 17, wherein A and B are —CH(R⁹)CH(R¹⁰); X is hydrogen, C₁-C₁₀ alkyl, —OH, or —NR¹¹R¹²; R¹, R¹¹, and R¹² are carboxymethyl; R² to R⁶, R⁹, and R¹⁰ are hydrogens.
